(19) United States
(12) Patent Application Publication
(10) Pub. No.: US 2025/0339068 A1
(43) Pub. Date: Nov. 6, 2025

(54) Blood Draw Syringe for Controlling Vacuum Pressure During Blood Collection

(71) Applicant: Becton, Dickinson and Company, Franklin Lakes, NJ (US)

(72) Inventors: Yiping Ma, Layton, UT (US); Weston F. Harding, Lehi, UT (US); Jonathan Karl Burkholz, Salt Lake City, UT (US); Daniel Blanchard, Bountiful, UT (US)

(21) Appl. No.: 19/192,577

(22) Filed: Apr. 29, 2025

Related U.S. Application Data
(60) Provisional application No. 63/642,188, filed on May 3, 2024.

(51) Int. Cl. A61B 5/15

(52) U.S. Cl. CPC A61B 5/150992; A61B 5/150244; A61B 5/150351

(57) ABSTRACT

Provided herein is a blood draw syringe connectable to a vascular access device for performing a blood draw. The blood draw syringe includes a syringe barrel and a plunger assembly. The plunger assembly includes a plunger rod and a stopper positioned at the distal end of the plunger rod, with the plunger assembly moveable within the syringe barrel to move the stopper between a starting position and a stop position, with a first volume provided in the chamber with the stopper at the starting position and a second volume provided in the chamber with the stopper at the stop position. The syringe is configured to enable pre-setting of the plunger assembly within the syringe barrel prior to a blood draw, with the stopper being positioned at the starting to provide a first volume that limits a maximum vacuum pressure in the syringe barrel during the blood draw.

### CROSS-REFERENCE TO RELATED APPLICATION

The present application claims priority to U.S. Provisional Patent Application No. 63/642,188 entitled “Blood Draw Syringe for Controlling Vacuum Pressure During Blood Collection” filed May 3, 2024, the entire disclosure of which is hereby incorporated by reference.

### BACKGROUND OF THE INVENTION

### Field of the Invention

The present disclosure relates generally to syringes for blood collection and, more particularly, to syringes that include features thereon for providing a controlled vacuum pressure in the syringe during blood draw, to mimic vacuum pressure in a vacuumed blood collection tube.

### Description of Related Art

Catheters are commonly used to administer fluids into and draw fluids (i.e., blood) out of the body, with patients in a variety of settings, including in hospitals and in home care, being administered such fluids or having blood drawn via a vascular access device (VAD) that includes such a catheter inserted into a patient's vascular system. A common VAD includes a plastic catheter that is inserted into a patient's vein, with a length of the catheter varying from a few centimeters when the VAD is a peripheral intravenous catheter (PIVC) to many centimeters when the VAD is a central venous catheter (CVC), as examples. A VAD may be indwelling for short term (days), moderate term (weeks), or long term (months to years).

When a VAD is used for purposes of a blood draw, a blood collection device or set may be attached to the VAD (with the catheter thereof indwelling in the patient) to enable collection of a blood specimen. In some cases, the blood collection device/set may be provided as a vacuumed blood collection tube such as a Vacutainer® device from Becton, Dickinson and Company, with the blood collection tube connected to the VAD via a luer-lock access device (LLAD) provided on the VAD. In other cases, the blood collection device/set may be provided as a syringe that is connected to the VAD, with a user actuating the syringe to facilitate collection of the blood specimen.

Vacuumed blood collection tubes (i.e., Vacutainers) are beneficial for use with a VAD for collection of a blood specimen in that they perform the blood draw with a controlled vacuum pressure, as determined by the vacuum pressure created/set during manufacturing of the vacuumed blood collection tube. This controlled vacuum pressure in the vacuumed blood collection tube helps to control potential hemolysis during the blood draw. However, drawbacks associated with use of a vacuumed blood collection tube include the requirement for a LLAD to be provided on the VAD, along with shelf-life limitations associated with the vacuumed blood collection tube.

The use of blood draw syringes with a VAD for collection of a blood specimen eliminate limitations associated with vacuumed blood collection tubes regarding requirements for a LLAD and a limited shelf-life; however, existing blood draw syringes introduce a separate set of challenges for performing of a blood draw. For example, when using blood draw syringes for collection of a blood specimen, a syringe draw rate during collection may be difficult for a user to control, with it recognized that an overly high draw rate can lead to increased mechanical hemolysis that may damage the collected blood sample—i.e., a vacuum pressure created in the blood draw syringe may be too high, leading to increased hemolysis.

Accordingly, a need exists in the art for a blood draw syringe useable with a VAD for collection of a blood sample, with the blood draw syringe designed to limit/control a vacuum pressure during the blood draw, such as by limiting the vacuum pressure to the level of a Vacutainer draw. It is also desirable for such a blood draw syringe to eliminate the impact of syringe draw rate on hemolysis, thus making the blood draw syringe more user friendly and standardizing the blood draw procedure.

### SUMMARY OF THE INVENTION

Provided herein is a blood draw syringe connectable to a vascular access device for performing a blood draw. The blood draw syringe comprises a syringe barrel having a distal end and a proximal end and that defines a chamber therein, and a fluid connector component at the distal end of the syringe barrel. The blood draw syringe also comprises a plunger assembly including a plunger rod having a proximal end and a distal end, and a stopper positioned at the distal end of the plunger rod that is insertable into the proximal end of the barrel, wherein the plunger assembly is moveable within the syringe barrel to move the stopper between a starting position and a stop position, with a first volume provided in the chamber between the stopper and the distal end of the syringe barrel with the stopper at the starting position and a second volume provided in the chamber between the stopper and the distal end of the syringe barrel with the stopper at the stop position, the second volume being greater than the first volume. The syringe barrel further includes a first marking provided on the syringe barrel indicating the starting position of the stopper and a second marking provided on the syringe barrel indicating the stop position of the stopper. With the plunger assembly moving proximally within the syringe barrel during a blood draw to move the stopper from the starting position to the stop position, a size of the first volume limits a vacuum pressure in the syringe barrel during the blood draw.

In some embodiments, the first marking comprises a first sub-marking provided on the syringe barrel indicating a first starting position of the stopper and a second sub-marking provided on the syringe barrel indicating a second starting position of the stopper.

In some embodiments, the blood draw syringe includes a clip attached to the plunger rod at a location that, when the clip abuts the proximal end of the syringe barrel, the stopper is at the starting position and aligned with the first marking.

In some embodiments, the clip is configured to be removable from the plunger rod, to enable a distal advancement of the plunger assembly fully into the syringe barrel.

In some embodiments, the blood draw syringe includes a stopcock provided in the syringe barrel and positioned at or adjacent the fluid connector component at the distal end of the syringe barrel, the stopcock actuatable between an open position and a closed position, to control a flow of fluid into the chamber of the syringe barrel.

In some embodiments, the first marking and the second marking comprise one or more of a line, a symbol, a picture, an icon, an alphabetical descriptor, or a numeric descriptor.

In some embodiments, a longitudinal positioning of the first marking and the second marking on the syringe barrel that indicate the starting position and the stop position of the stopper, respectively, are selected to set the first volume and the second volume and, in turn, set a maximum desired vacuum pressure in the syringe barrel during the blood draw.

In some embodiments, the maximum desired vacuum pressure is a vacuum pressure equal to or less than a vacuum pressure in a vacuumed blood collection tube having a same draw volume as a draw volume of the blood draw syringe.

Also provided herein is a blood draw syringe connectable to a vascular access device for performing a blood draw. The blood draw syringe comprises a syringe barrel having a distal end and a proximal end and that defines a chamber therein, and a fluid connector component at the distal end of the syringe barrel. The blood draw syringe also comprises a plunger assembly including a plunger rod having a proximal end and a distal end, and a stopper positioned at the distal end of the plunger rod that is insertable into the proximal end of the barrel, wherein the plunger assembly is moveable within the syringe barrel to move the stopper between a starting position and a stop position, with a first volume provided in the chamber between the stopper and the distal end of the syringe barrel with the stopper at the starting position and a second volume provided in the chamber between the stopper and the distal end of the syringe barrel with the stopper at the stop position, the second volume being greater than the first volume. The plunger rod further includes a stop arm affixed thereto at or adjacent the distal end of the plunger rod, the stop arm including a proximal end affixed to the plunger rod and a free distal end having a catch formed thereon configured to engage the proximal end of the syringe barrel when the plunger assembly is in the starting position. The stop arm is at an initial angle when engaged with the proximal end of the syringe barrel and is configured to rotate to a second angle when disengaged from the proximal end of the syringe barrel.

In some embodiments, with the plunger assembly in the starting position, the stop arm prevents further distal advancement of the plunger assembly into the syringe barrel.

In some embodiments, the catch is disengaged from the proximal end of the syringe barrel upon a proximal movement of the plunger assembly.

In some embodiments, with the stop arm rotated to the second angle, the plunger assembly may be distally advanced into the syringe barrel, until the stopper contacts the distal end of the syringe barrel.

In some embodiments, when rotated to the second angle, the stop arm is rotated toward the plunger rod or rotated away from the plunger rod.

In some embodiments, with the plunger assembly moving proximally within the syringe barrel during a blood draw to move the stopper from the starting position to the stop position, a size of the first volume limits a vacuum pressure in the syringe barrel during the blood draw.

Also provided herein is a system for performing a blood draw. The system comprises a vascular access device including a catheter comprising a catheter distal end and a catheter proximal end, a catheter adapter including a distal adapter end and a proximal adapter port and defining a lumen therein, wherein the catheter proximal end is secured to the distal adapter end, and an extension set extending from the catheter adapter and in fluid communication with the catheter via the lumen of the catheter adapter, with the extension set comprising an extension tube having a distal end coupled to the catheter adapter and a proximal end having a connector thereon. The system also comprises a syringe connected to the vascular access device. The syringe includes a syringe barrel having a distal end and a proximal end and that defines a chamber therein, and a fluid connector component at the distal end of the barrel that is configured to engage the connector, to fluidly connect the syringe to the vascular access device. The syringe also includes a plunger assembly including a plunger rod having a proximal end and a distal end, and a stopper positioned at the distal end of the plunger rod that is insertable into the proximal end of the barrel, wherein the plunger assembly is moveable within the syringe barrel between a starting position and a stop position, with a first volume provided in the chamber between the stopper and the distal end of the syringe barrel with the stopper at the starting position and a second volume provided in the chamber between the stopper and the distal end of the syringe barrel with the stopper at the stop position, the second volume being greater than the first volume. The syringe is configured to enable pre-setting of the plunger assembly within the syringe barrel prior to a blood draw, with the stopper positioned at the starting position, thereby limiting a vacuum pressure in the syringe barrel during the blood draw.

In some embodiments, the syringe barrel includes a first marking provided on the syringe barrel indicating the starting position of the stopper and a second marking provided on the syringe barrel indicating the stop position of the stopper. With the plunger assembly moving proximally within the syringe barrel during a blood draw to move the stopper from the starting position to the stop position, a size of the first volume limits a vacuum pressure in the syringe barrel during the blood draw.

In some embodiments, the syringe further includes a clip attached to the plunger rod at a location that, when the clip abuts the proximal end of the syringe barrel, the stopper is at the starting position and aligned with the first marking, and wherein the clip is removable from the plunger rod, to enable a distal advancement of the plunger assembly fully into the syringe barrel.

In some embodiments, the syringe further includes a stopcock provided in the syringe barrel and positioned at or adjacent the fluid connector component at the distal end of the syringe barrel, the stopcock actuatable between an open position and a closed position, to control a flow of fluid into the chamber of the syringe barrel.

In some embodiments, the plunger rod includes a stop arm affixed thereto at or adjacent the distal end of the plunger rod, the stop arm including a proximal end affixed to the plunger rod and a free distal end having a catch formed thereon configured to engage the proximal end of the syringe barrel when the plunger assembly is in the starting position. The stop arm is at an initial angle when engaged with the proximal end of the syringe barrel and is configured to rotate to a second angle when disengaged from the proximal end of the syringe barrel.

Also provided herein is a method of performing a blood draw. The method includes positioning the plunger assembly within the syringe barrel so that the stopper is at the starting position, thereby providing the first volume in the chamber between the stopper and the distal end of the syringe barrel. The method also includes coupling the syringe to the vascular access device, via mating of the fluid connector component with the connector, and proximally retracting the plunger assembly within the syringe barrel to move the stopper from the starting position to the stop position, so as to draw a blood sample into the chamber of the syringe barrel. In positioning the stopper at the starting position and providing the first volume prior to initiation of the blood draw, a vacuum pressure created in the syringe barrel during the blood draw is limited.

In some embodiments, a longitudinal positioning of the starting position and the stop position of the stopper, respectively, are selected to set the first volume and the second volume and, in turn, set a maximum desired vacuum pressure in the syringe barrel during the blood draw.

In some embodiments, the maximum desired vacuum pressure is a vacuum pressure equal to or less than a vacuum pressure in a vacuumed blood collection tube having a same draw volume as a draw volume of the blood draw syringe.

Also provided herein is an adapter-syringe assembly connectable to a vascular access device for performing a blood draw. The adapter-syringe assembly comprises a blood draw syringe and an adapter. The blood draw syringe including a syringe barrel having a distal end and a proximal end and that defines a chamber therein, and a fluid connector component at the distal end of the syringe barrel, along with and a plunger assembly including a plunger rod having a proximal end and a distal end, and a stopper positioned at the distal end of the plunger rod that is insertable into the proximal end of the syringe barrel, wherein the plunger assembly is moveable within the syringe barrel to move the stopper between a starting position and a stop position, with blood draw volume provided in the chamber between the stopper and the distal end of the syringe barrel with the stopper at the stop position. The adapter is coupled to the blood draw syringe and includes an adapter body defining an internal adapter volume, a distal connector engageable with the vascular access device, and a proximal connector engageable with the fluid connector component at the distal end of the syringe barrel, wherein a size of the internal adapter volume limits a vacuum pressure in the syringe barrel during the blood draw.

In some embodiments, the size of the internal adapter volume of the adapter body is selected based on the blood draw volume to set a maximum desired vacuum pressure in the syringe barrel during the blood draw.

In some embodiments, the maximum desired vacuum pressure is a vacuum pressure equal to or less than a vacuum pressure in a vacuumed blood collection tube having a same blood draw volume as a blood draw volume of the blood draw syringe.

In some embodiments, the adapter body comprises an expandable or extendable adapter body configurable to a shortened configuration and a lengthened configuration, with the internal adapter volume comprising a first internal adapter volume with the extendable adapter body in the shortened configuration and a second internal adapter volume with the extendable adapter body in the lengthened configuration.

In some embodiments, the adapter further comprises a stopcock provided in the adapter body, the stopcock actuatable between an open position and a closed position, to control a flow of fluid through the adapter and into the syringe barrel.

Also provided herein is an adapter connectable to a vascular access device and a syringe for performing a blood draw. The adapter includes an adapter body defining an internal adapter volume, a distal connector engageable with the vascular access device, and a proximal connector engageable with the syringe, wherein a size of the internal adapter volume limits a vacuum pressure in the syringe during the blood draw.

In some embodiments, the size of the internal adapter volume of the adapter body is selected based on the blood draw volume to set a maximum desired vacuum pressure in the syringe during the blood draw.

In some embodiments, the maximum desired vacuum pressure is a vacuum pressure equal to or less than a vacuum pressure in a vacuumed blood collection tube having a same blood draw volume as a blood draw volume of the syringe.

In some embodiments, the adapter body comprises an expandable or extendable adapter body configurable to a shortened configuration and a lengthened configuration, with the internal adapter volume comprising a first internal adapter volume with the extendable adapter body in the shortened configuration and a second internal adapter volume with the extendable adapter body in the lengthened configuration.

In some embodiments, the adapter further comprises a stopcock provided in the adapter body, the stopcock actuatable between an open position and a closed position, to control a flow of fluid through the adapter and into the syringe.

Also provided herein is a system for performing a blood draw. The system comprises a vascular access device including a catheter comprising a catheter distal end and a catheter proximal end, a catheter adapter including a distal adapter end and a proximal adapter port and defining a lumen therein, wherein the catheter proximal end is secured to the distal adapter end, and an extension set extending from the catheter adapter and in fluid communication with the catheter via the lumen of the catheter adapter, the extension set comprising an extension tube having a distal end coupled to the catheter adapter and a proximal end having a needle set thereon that includes a holder and a needle. The system also includes an adapter-syringe assembly connected to the vascular access device. The adapter-syringe assembly comprises a syringe and an adapter. The syringe includes a syringe barrel having a distal end and a proximal end and that defines a chamber therein and a plunger assembly including a plunger rod having a proximal end and a distal end, and a stopper positioned at the distal end of the plunger rod that is insertable into the proximal end of the syringe barrel, wherein the plunger assembly is moveable within the syringe barrel to move the stopper between a starting position and a stop position, with blood draw volume provided in the chamber between the stopper and the distal end of the syringe barrel with the stopper at the stop position. The adapter is coupled to the blood draw syringe and includes an adapter body defining an internal adapter volume, a proximal end comprising a proximal connector engageable with the distal end of the syringe barrel, and a distal end comprising a rubber plug that seals the distal end, wherein the distal end is configured to be received within the holder, with the needle piercing the rubber plug, and wherein with the adapter coupled to the blood draw syringe, the plunger assembly may be retracted to move the stopper to the stop position, thereby creating a vacuum pressure within the adapter and the chamber of the syringe barrel that draws blood into the chamber upon piercing of the rubber plug by the needle.

In some embodiments, the vacuum pressure is a vacuum pressure equal to or less than a vacuum pressure in a vacuumed blood collection tube having a same draw volume as a draw volume of the blood draw syringe.

In some embodiments, the adapter-syringe assembly further comprises a clip attached to the plunger rod that retains the plunger assembly at a retracted position, with the stopper at the stop position, the clip configured to be removable from the plunger rod, to enable a distal advancement of the plunger assembly fully into the syringe barrel.

Also provided herein is a blood draw syringe connectable to a vascular access device for performing a blood draw. The blood draw syringe comprises a syringe barrel having a distal end and a proximal end and that defines a chamber therein, and a fluid connector component at the distal end of the syringe barrel. The blood draw syringe also comprises a plunger assembly including a plunger rod having a proximal end and a distal end, and a stopper positioned on the plunger rod that is insertable into the proximal end of the barrel, wherein the plunger assembly is moveable within the syringe barrel to move the stopper between a starting position and a stop position, with a first volume provided in the chamber between the stopper and the distal end of the syringe barrel with the stopper at the starting position and a second volume provided in the chamber between the stopper and the distal end of the syringe barrel with the stopper at the stop position, the second volume being greater than the first volume. The plunger assembly further comprises an air chamber or air pocket defined at least partially by at least one of the plunger rod and the stopper, with the air chamber or air pocket forming part of the first volume. With the plunger assembly moving proximally within the syringe barrel during a blood draw to move the stopper from the starting position to the stop position, a size of the first volume limits a vacuum pressure in the syringe barrel during the blood draw.

In some embodiments, the air chamber or air pocket comprises an air chamber defined by the distal end of the plunger rod, and wherein the stopper includes a channel formed therethrough that is aligned with the air chamber, so that the air chamber is fluidly connected to the chamber of the syringe barrel.

In some embodiments, the air chamber or air pocket comprises an air chamber defined by the stopper, and wherein the stopper includes a hollow protrusion extending out proximally from a remainder of the stopper and that defines the air chamber, wherein the protrusion is received within another chamber formed in the distal end of the plunger rod, and wherein the air chamber of the stopper is fluidly connected to the chamber of the syringe barrel.

In some embodiments, the blood draw syringe further comprises a vent plug positioned on the stopper and separating the chamber of the syringe barrel from the air chamber, wherein the vent plug is configured to be air permeable but impervious to fluids.

In some embodiments, the air chamber or air pocket comprises an air pocket, wherein the stopper comprises an elongated stopper including proximal ribs and a distal rib, separated by an inter-rib gap, the proximal ribs configured to form a tight seal with the syringe barrel and the distal rib including a plurality of channels arranged about a circumference thereof, and wherein the air pocket is formed in a volume defined between the syringe barrel and the inter-rib gap region of the stopper, with the air pocket being fluidly connected with the chamber of the syringe barrel via the channels formed in the distal rib.

In some embodiments, the air chamber or air pocket comprises an air pocket, wherein the stopper comprises a proximal stopper and the plunger assembly further comprises a distal stopper spaced apart longitudinally from the proximal stopper and attached to the distal end of the plunger rod, the distal stopper comprising one or more annular ribs thereon each including a plurality of channels arranged about a circumference thereof, and wherein the air pocket is defined by the syringe barrel and a volume between the proximal stopper and the distal stopper, with the air pocket being fluidly connected with the chamber of the syringe barrel via the channels formed in the one or more annular ribs of the distal stopper.

### BRIEF DESCRIPTION OF THE DRAWINGS

FIG. 1 is a perspective view of a vascular access device and blood draw syringe for performing of a blood draw, according to an embodiment of the disclosure;

FIG. 2 is an exploded view of the syringe of FIG. 1;

FIGS. 3A-3C are side perspective views of the syringe of FIG. 1 in various operational states, according to an embodiment of the disclosure;

FIG. 4 is a side perspective view of the syringe of FIG. 1, according to another embodiment of the disclosure;

FIGS. 5A-5C are side perspective views of the syringe of FIG. 1 in various operational states, according to another embodiment of the disclosure;

FIGS. 6A-6C are side perspective views of the syringe of FIG. 1 in various operational states, according to another embodiment of the disclosure;

FIGS. 7A-7C are side perspective views of the syringe of FIG. 1 in various operational states, according to another embodiment of the disclosure;

FIG. 8A-8C are side perspective views of the syringe of FIG. 1 in various operational states, according to another embodiment of the disclosure;

FIGS. 9A-9C are side perspective views of an adapter-syringe assembly useable with the vascular access device of FIG. 1, with the adapter-syringe assembly shown in various operational states, according to an embodiment of the disclosure;

FIGS. 10A and 10B are side perspective views of an adapter having an extendable/expandable adapter body, according to an embodiment of the disclosure;

FIGS. 11A-11C are side perspective views of an adapter-syringe assembly useable with the vascular access device of FIG. 1, with the adapter-syringe assembly shown in various operational states, according to another embodiment;

FIG. 12 is a perspective view of a vascular access device and adapter-syringe assembly for performing of a blood draw, according to another embodiment of the disclosure;

FIGS. 13A-13C are side perspective views of the adapter-syringe assembly of FIG. 12 shown in various operational states, according to another embodiment of the disclosure;

FIG. 14 is a side cross-sectional view of a plunger assembly useable with the syringe of FIG. 2, according to another embodiment of the disclosure;

FIG. 15 is a side cross-sectional view of a plunger assembly useable with the syringe of FIG. 2, according to another embodiment of the disclosure;

FIG. 16 is a side cross-sectional view of a plunger assembly useable with the syringe of FIG. 2, according to another embodiment of the disclosure;

FIG. 17 is a cross-section of the plunger assembly of FIG. 16 taken along line 17-17;

FIG. 18 is a side cross-sectional view of a plunger assembly useable with the syringe of FIG. 2, according to another embodiment of the disclosure; and

FIG. 19 is a cross-section of the plunger assembly of FIG. 18 taken along line 19-19.

### DESCRIPTION OF THE INVENTION

The following description is provided to enable those skilled in the art to make and use the described aspects contemplated for carrying out the invention. Various modifications, equivalents, variations, and alternatives, however, will remain readily apparent to those skilled in the art. Any and all such modifications, variations, equivalents, and alternatives are intended to fall within the spirit and scope of the present disclosure.

For the purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawings. However, it is to be understood that the invention may assume various alternative variations, except where expressly specified to the contrary. It is also to be understood that the specific devices illustrated in the attached drawings, and described in the following specification, are simply exemplary aspects of the invention. Hence, specific dimensions and other physical characteristics related to the aspects disclosed herein are not to be considered as limiting.

In the present disclosure, the distal end of a component or of a device means the end furthest away from the hand of the user and the proximal end means the end closest to the hand of the user, when the component or device is in the use position, i.e., when the user is holding a blood draw device in preparation for or during use. Similarly, in this application, the terms “in the distal direction” and “distally” mean in the direction toward an access connector portion of the fluid transfer device, and the terms “in the proximal direction” and “proximally” mean in the direction opposite the direction of the connector.

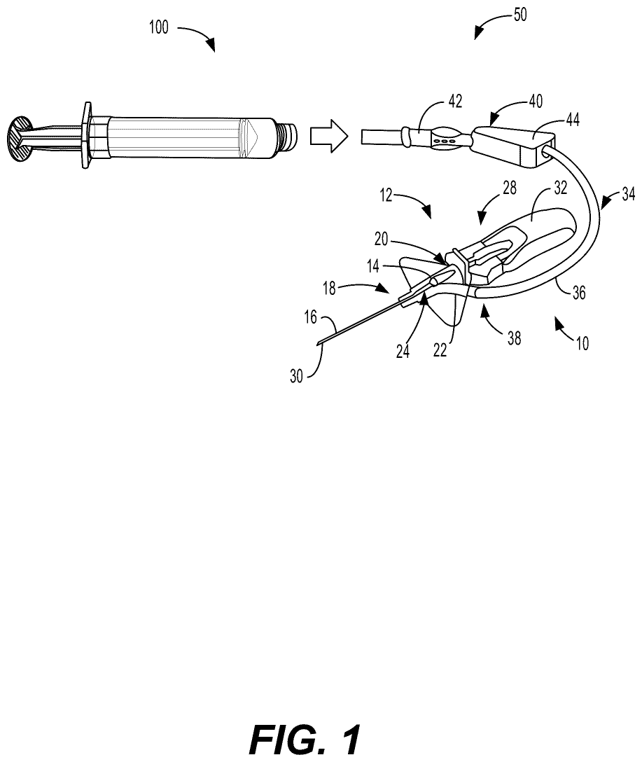

Referring to FIG. 1, shown is a non-limiting embodiment of a vascular access device 10 for facilitating blood draw from a patient. As shown in FIG. 1, VAD 10 is provided as a catheter assembly 12 that provides access to the vasculature of a patient. According to embodiments of the disclosure, the VAD 10 may have any of a number of suitable configurations, including being constructed/arranged as an integrated catheter assembly or a non-integrated catheter assembly, with non-limiting examples including the BD Nexiva™ Closed IV Catheter system, the BD Intima™ Catheter system,, or other suitable vascular access devices.

As shown in FIG. 1, the VAD 10 comprises a catheter assembly 12 that includes a catheter adapter 14 and associated catheter 16. The catheter adapter 14 may include a distal end 18 and a proximal end 20. In some embodiments, the catheter adapter 14 may include an additional adapter port 22 that may disposed between the distal end 18 and the proximal end 20 or disposed at the proximal end 20. The catheter adapter 14 may include a first lumen 24 extending through the distal end 18 and the proximal end 20, and the lumen 24 may be sealed at proximal end 20 of catheter adapter 14, such as by a split-septum connector or self-healing septum connector (not shown). The catheter 16 extends from the distal end 18 of catheter adapter 14 and may be configured as a PIVC that is placed into a vein of the patient, with a distal end or tip of the catheter 16 positioned appropriately within the vein to enable a blood draw from the patient. Catheter 16 may be formed of any suitable material and may be of any useful length, as known to those of skill in the art.

In some non-limiting embodiments or aspects, a needle subassembly 28 may be provided at the proximal end 20 of the catheter adapter 14 with the needle subassembly 28 including a needle 30 that extends through the catheter adapter 14 (i.e., through lumen 24 thereof) and into the catheter 16, in a catheter-over-needle arrangement, for example. The needle subassembly 28 may comprise the needle 30 and a housing 32. The housing 32 may engage the proximal end 20 of the catheter adapter 14 and may be configured to control movement of the needle 30 relative to the catheter 16 and/or catheter adapter 14, including during insertion of the needle 30 into the vasculature of a patient and during a subsequent withdrawal of the needle 30 out from the catheter 16 and out through the catheter adapter 12.

In some non-limiting embodiments or aspects, the catheter assembly 12 may include an extension set 34 coupled to the port 22. The extension set 34 may be used to provide a fluid path from the VAD 10 to enable blood aspiration. The extension set 34 may include an extension tube 36 having a distal end 38 and a proximal end 40. The distal end 38 of extension tube 36 may be coupled to port 22, while the proximal end 40 of extension tube 36 may include an adapter or connector 42. In some embodiments, the connector 42 may comprise a luer connection or other needleless access connector. In some embodiments, a clamp 44 may be provided on extension tube 36 between the distal and proximal ends 38, 40 thereof, with the clamp 44 being selectively actuatable to occlude a fluid flow through the extension tube 36.

As further shown in FIG. 1, a blood draw syringe 100 (hereafter “syringe 100”) may be provided that is coupleable to the VAD 10 to enable performing of a blood draw-with the VAD and syringe 100 collectively providing a system 50 for blood collection. The syringe may be connected to the extension set via connector 42, such as via mating of corresponding luer connections of the connector 42 and the syringe 100, as explained in further detail below. With the syringe 100 coupled to the VAD 10, the syringe 100 may then be actuated by a user to aspirate blood into the syringe 100, for collection of a blood sample.

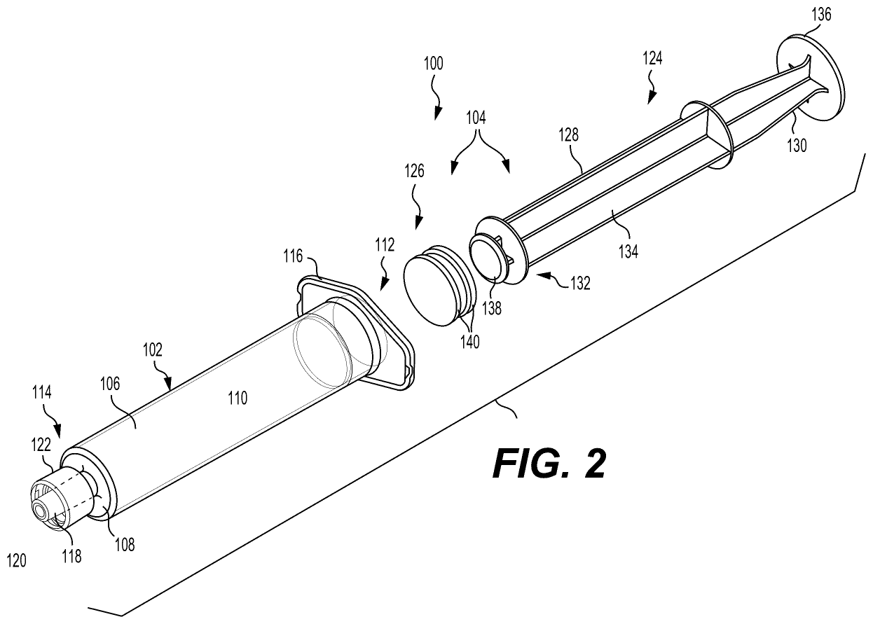

Referring to FIG. 2, the syringe 100 is shown in more detail according to a non-limiting embodiment. The syringe 100 generally includes a syringe barrel 102 and a plunger assembly 104. The plunger assembly 104 is axially movable within the syringe barrel 102 from an advanced position to a retracted position, to facilitate a blood draw from a patient, for example.

The syringe barrel 102 is formed of a generally cylindrical outer wall 106 and an end wall 108 that collectively define a chamber 110 for retaining fluid therein. The syringe barrel 102 includes an open proximal end 112 configured to receive the plunger assembly 104 therein and a distal end 114 at which end wall 108 is positioned. The proximal end 112 of the syringe barrel 102 may include a flange 116 to facilitate handling and positioning of the syringe 100 and to maintain the relative position of the syringe barrel 102 with respect to the plunger assembly 104 during medication administration. At the distal end 114, a tip 118 extends distally outward from the end wall 108 and defines a lumen 120 in fluid communication with the chamber 110. A fluid connector component 122 is provided on the tip 118 that is coupleable to a corresponding connector (e.g., connector 42) to which the syringe 100 is to be brought into engagement for performing of a blood draw. In some embodiments, fluid connector component 122 is configured as a luer adapter configured to engage a corresponding luer connection of connector 42, on the VAD 10.

The plunger assembly 104 of syringe 100 is formed of an elongate plunger rod 124 and a plunger head or stopper 126. The plunger rod 124 may include a main body 128 extending between a plunger proximal end 130 and a plunger distal end 132. In some embodiments, the main body 128 may include a plurality of elongate vanes or walls 134 extending axially along a length thereof between the plunger proximal end 130 and the plunger distal end 132. A thumb press 136 is positioned at the plunger proximal end 130 that may be engaged by a thumb (or other finger) of the user to apply a distally directed force to the plunger assembly 104 to move the plunger rod 124 with respect to the syringe barrel 102. In some embodiments, a flanged extension member 138 (e.g., disc-shaped flange) is positioned at the plunger distal end 132 that is configured to mate with the stopper 126.

The stopper 126 of plunger assembly 104 is positioned at the plunger distal end 132 so as to be movable along with the plunger rod 124 within the chamber 110 of syringe barrel 102. The stopper 126 may be made from a material that is different from the material of the plunger rod 124 and that is capable of forming a tight seal with the syringe barrel 102 as it is moved therethrough. In some embodiments, the stopper 126 includes a receptacle (not shown) formed therein that is sized and configured to receive the flanged extension member 138 of plunger rod 124, with the flanged extension member 138 coupling with the receptacle via a press fit connection, for example, to secure the stopper 126 to the plunger rod 124, although it can be appreciated that the stopper 126 and plunger distal end 132 can be secured by other techniques known in the art. The stopper 126 may further include a pair of spaced-apart annular flanges 140 formed thereon (i.e., O-rings) that form a tight seal with the cylindrical outer wall 106 of syringe barrel 102.

According to embodiments of the disclosure, the syringe 100 is configured to provide for collection of a blood sample in a controlled manner, so as to reduce mechanical hemolysis during the blood draw and provide for a faster blood fill time in obtaining the sample. In particular, the syringe 100 may be pre-set/pre-configured with the stopper 126 at a desired location within syringe barrel 102 prior to initiation of a blood draw sequence, with the pre-set positioning of the stopper 126 functioning to control a vacuum pressure within the syringe 100 during the blood draw. That is, it is recognized that start and stop positions of the stopper 126 in syringe 100 correspond to an initial volume (V1) and a final volume (V2) at the start and stop of the blood draw, as determined by a position of stopper 126 of the syringe 100, such that V2−V1 is the desired blood draw volume. According to the ideal gas law, the vacuum pressure within the syringe 100 at each of the volumes is dependent on the magnitude/size of the volume, with the product of pressure and volume being equal at the initial and final volumes according to:

$$P 2 * V 2 = P 1 * V 1 ,$$

where P2 is a second pressure within the syringe 100 with the syringe 100 at the stop position and the volume at the final volume V2, and P1 is a first pressure (e.g. ambient pressure) within the syringe 100 with the syringe 100 at the start position and the volume at the initial volume V1. Thus, knowing the relationship between pressure and volume, a pre-setting of the initial volume V1 in syringe 100 can ensure that the maximum vacuum pressure within the syringe 100 during a blood draw is controlled to a desired level. In some embodiments, the maximum vacuum pressure P2 may be controlled so that it matches the maximum vacuum pressure that is set/provided within a vacuumed blood collection tube (i.e., a Vacutainer) of similar draw volume, as explained in further detail below.

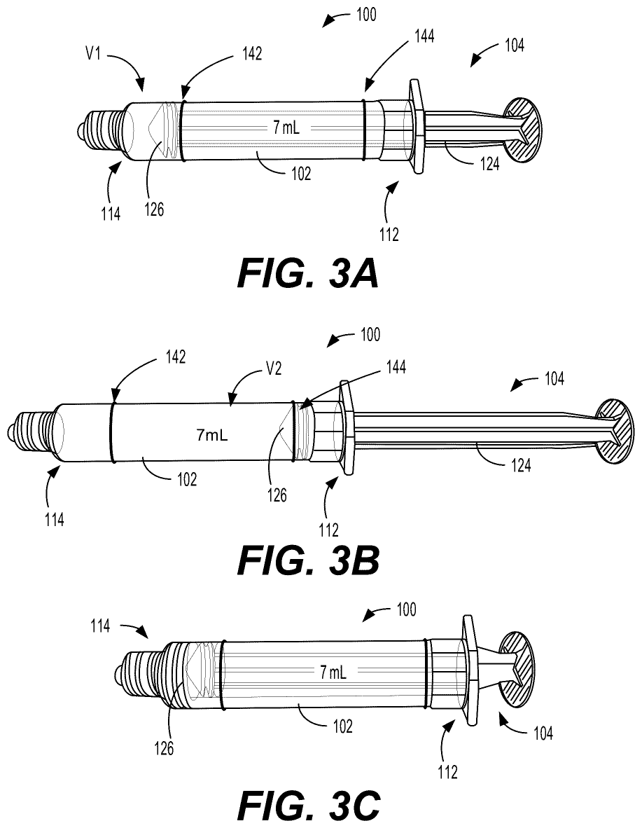

Various embodiments of a syringe 100 that provide for collection of a blood sample in a controlled manner are provided in FIGS. 3-8, according to aspects of the disclosure. As described above, each of these syringes 100 is configured to be pre-set/pre-configured to a desired collection volume prior to initiation of a blood draw sequence, with the pre-set volume functioning to control a vacuum pressure within the syringe 100 during the blood draw.

Referring now to FIGS. 3A-3C, and with continued reference to FIG. 2, a syringe 100 is shown according to one embodiment of the disclosure. In order to provide for a pre-setting/pre-configuring of the syringe 100 to a desired collection volume and at a desired maximum vacuum pressure during blood draw, the barrel 102 of syringe 100 includes a plurality of markings thereon. A first marking 142 is provided on syringe barrel 102 that indicates a starting position for the plunger assembly 104 (i.e., starting position for stopper 126), as shown in FIG. 3A, and a second marking 144 is provided on syringe barrel 102 that indicates a stop position for the plunger assembly 104 (i.e., stop position for stopper 126) after completion of a blood draw, as shown in FIG. 3B. According to various embodiments, the markings 142, 144 may be considered/defined to include one or more of the following: a line, symbol, a picture, an icon, an alphabetical descriptor, a numeric descriptor, or another suitable indicator marking. In some embodiments, the markings 142, 144 may be stamped, embossed, laser-drilled, marked, scribed, or created in other suitable manners.

With the stopper 126 at the first marking 142, a first volume V1 (see FIG. 3A) is defined within syringe barrel 102 between the distal end 114 of the syringe barrel 102 and the stopper 126 and, with the stopper 126 at the second marking 144, a second volume V2 (see FIG. 3B) is defined within syringe barrel 102 between the distal end 114 of the syringe barrel 102 and the stopper 126. A desired draw volume for the syringe 100 is thus defined as the second volume V2 minus the first volume V1 (V_draw=V2−V1). As an example, if the draw volume of the syringe 100 is 7 mL, V1 may be selected as 0.6 mL and V2=7+0.6=7.6 mL.

As previously described, the maximum vacuum pressure provided in syringe 100 during a blood draw may be set/controlled via positioning of the stopper 126 at a desired initial location, i.e., setting the initial volume V1. Using the ideal gas law, a maximum pressure during blood draw can be defined as:

$$P 2 = P 1 * V 1 / V 2$$

Knowing a desired P2 value, an initial volume V1 may be selected that ensures that P2 is set at a desired value, with the first marking 142 enabling a simple positioning of the plunger assembly 104 (and stopper 126) within syringe barrel 102 to provide this initial volume. In some embodiments, the maximum pressure P2 may be selected so that the syringe 100 has a same maximum vacuum pressure as the maximum vacuum pressure within a Vacutainer® for the same draw volume, to ensure that hemolysis during blood draw is similar to that during a Vacutainer blood draw. Thus, for above example where V1 is selected as 0.6 mL and V2=7+0.6=7.6 mL, P2 may be set at a level of P2=101 kPa (for P1 as the ambient pressure at sea level)×0.6 (V1)/7.6 (V2)=8 kPa (absolute), which matches the maximum vacuum pressure within a 13×100 size Vacutainer tube for 7 mL draw volume.

For the syringe 100 as illustrated in FIGS. 3A-3C, performing of a blood draw may thus include steps of: (1) pulling the plunger assembly 104 proximally to set the stopper 126 at the starting position, at the first marking 142, as shown in FIG. 3A; (2) connecting the syringe 100 to the VAD 10; (3) pulling the plunger assembly 104 proximally to the stop position, at the second marking 144, as shown in FIG. 3B, to collect the blood sample; and (4) at the desired time, pushing the plunger assembly 104 distally to the distal end 114 of the syringe barrel 102 to dispense the blood sample to a specimen container, as shown in FIG. 3C.

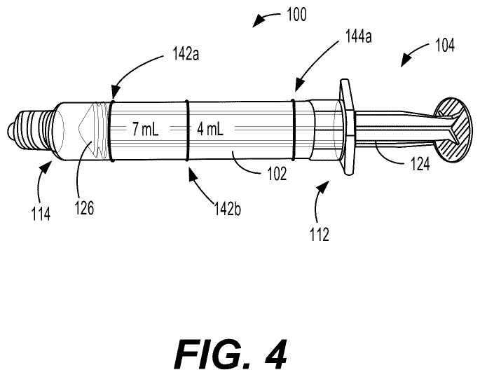

Referring now to FIG. 4, the syringe 100 of FIGS. 3A-3C may be modified to include an additional marking thereon, according to another embodiment of the disclosure. That is, a pair of first marking 142a, 142b (i.e., a first sub-marking 142a and a second sub-marking 142b) may be provided on syringe barrel 102 to enable the syringe 100 to be used for different blood draw volumes. For example, if it is desired to enable a draw volume in syringe 100 of 7 mL and 4 mL, a first sub-marking 142a may be provided on syringe barrel 102 indicating a starting position for the plunger assembly 104 (i.e., starting position for stopper 126) relative to the stop position for the 7 mL draw, and a second sub-marking 142b may be provided on syringe barrel 102 indicating a starting position for the plunger assembly 104 (i.e., starting position for stopper 126) relative to the stop position for the 4 mL draw. Each of the start positions at the first and second sub-markings 142a, 142b may be selected to provide an initial volume V1 that sets the maximum vacuum pressure P2 in syringe 100 at a desired level, as described in detail above.

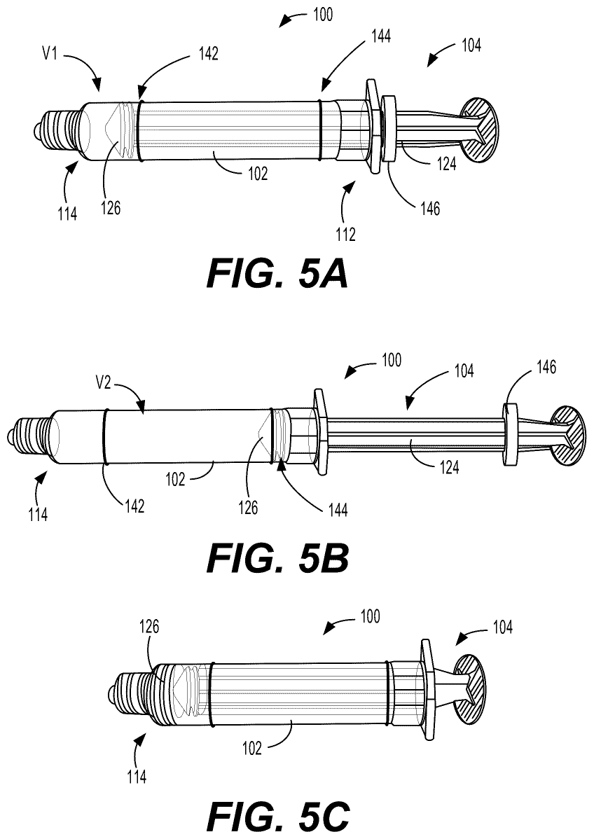

Referring now to FIGS. 5A-5C, and with continued reference to FIG. 2, a syringe 100 is shown according to another embodiment of the disclosure. The syringe 100 is identical to the syringe of FIGS. 3A-3C (with the barrel 102 including a first marking 142 and a second marking 144), except that a removable clip 146 is provided on the syringe 100 to ensure that plunger assembly 104 is retained at the starting position prior to use of the syringe 100 for a blood draw. The removable clip 146 is attached to the plunger rod 124 such that the forward-most position of the plunger assembly 104 with the clip 146 in place corresponds to the starting position. Inclusion of the removable clip 146 beneficially eliminates one step in the blood draw process—i.e., it is no longer necessary for an end user to pull the plunger assembly 104 proximally to the starting position, as this may be done during manufacturing, with the clip 146 affixed to the plunger rod 124 at a position that—as the plunger assembly 104 is pushed distally into syringe barrel 102 during assembly—the clip 146 engages the proximal end 112 of the syringe barrel 102 when the plunger assembly 104 is positioned at the desired starting position, with the clip 146 limiting the amount/length that plunger assembly 104 can be inserted into the syringe barrel 102 and defining the starting position of the plunger assembly 104 (and stopper 126). Then, upon completion of the blood draw, the clip 146 can be removed from the plunger rod 124, to allow the plunger assembly 104 to be advanced distally a full distance within syringe barrel 102, so that the collected blood may be dispensed into a specimen container.

For the syringe 100 as illustrated in FIGS. 5A-5C, performing of a blood draw may thus include steps of: (1) connecting the syringe 100 to the VAD 10, with the plunger assembly 104 pre-arranged at the first marking 142, at the starting position, as shown in FIG. 5A; (2) pulling the plunger assembly 104 proximally to the second marking 144, at the stop position, as shown in FIG. 5B, to collect the blood sample; and (3) at the desired time, removing the clip 146 from plunger rod 124 and pushing the plunger assembly 104 distally to the distal end 114 of the syringe barrel 102 to dispense the blood sample to a specimen container, as shown in FIG. 5C.

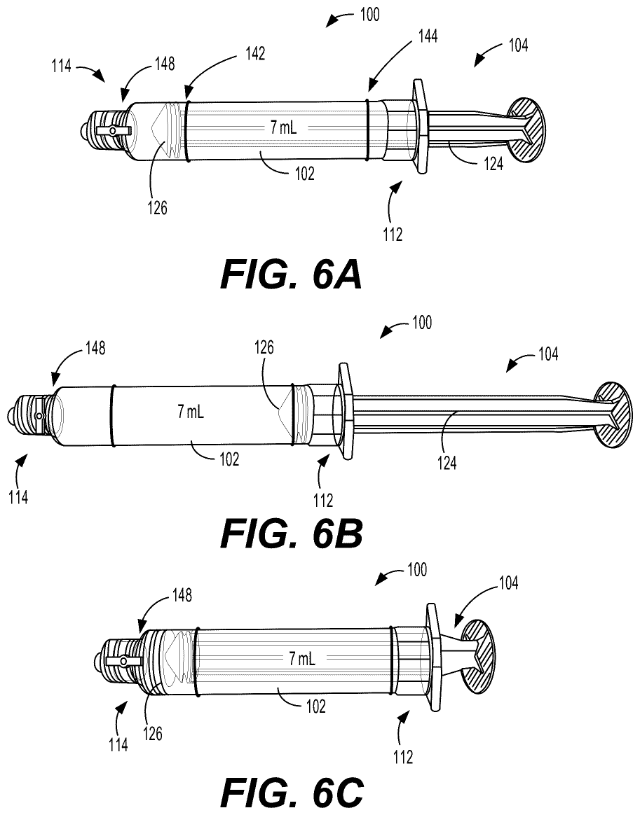

Referring now to FIGS. 6A-6C, and with continued reference to FIG. 2, a syringe 100 is shown according to another embodiment of the disclosure. In the syringe 100, first and second markings 142, 144 may again be provided on the syringe barrel 102 corresponding to a starting position and a stop position of the plunger assembly 104. Additionally, in the illustrated embodiment, the syringe 100 also includes a stopcock 148 integrated therein that may be actuated between an open position and a closed position-with the stopcock provided at/near the distal end 114 of the syringe barrel 102, such as in/on tip 118. The stopcock 148 may be in the open position when the syringe 100 is manufactured, allowing the plunger assembly 104 to be drawn back to a starting position during that sets the initial volume V1 at a desired amount. The stopcock 148 may then be actuated to the closed position once the syringe 100 is attached to a VAD 10, but before the plunger assembly 104 is drawn to the stop position. Actuating of the stopcock 148 to the closed position prior to pulling of the plunger assembly 104 back toward the stop position will allow the syringe 100 to reach a maximum vacuum pressure possible before any blood flows into the syringe 100, which would reduce the maximum vacuum in the syringe 100 while blood is being drawn into the syringe barrel 102.

For the syringe 100 as illustrated in FIGS. 6A-6C, performing of a blood draw may thus include steps of: (1) pulling the plunger assembly 104 proximally to set the stopper 126 at the starting position, at the first marking 142, as shown in FIG. 6A; (2) connecting the syringe 100 to the VAD 10; (3) actuating the stopcock 148 to the closed position upon connection of the syringe 100 to the VAD 10; (4) pulling the plunger assembly 104 proximally to the stop position, at the second marking 144, as shown in FIG. 6B; (5) actuating the stopcock 148 to the open position to collect the blood sample; and (6) at the desired time, pushing the plunger assembly 104 distally to the distal end 114 of the syringe barrel 102 to dispense the blood sample to a specimen container, as shown in FIG. 6C.

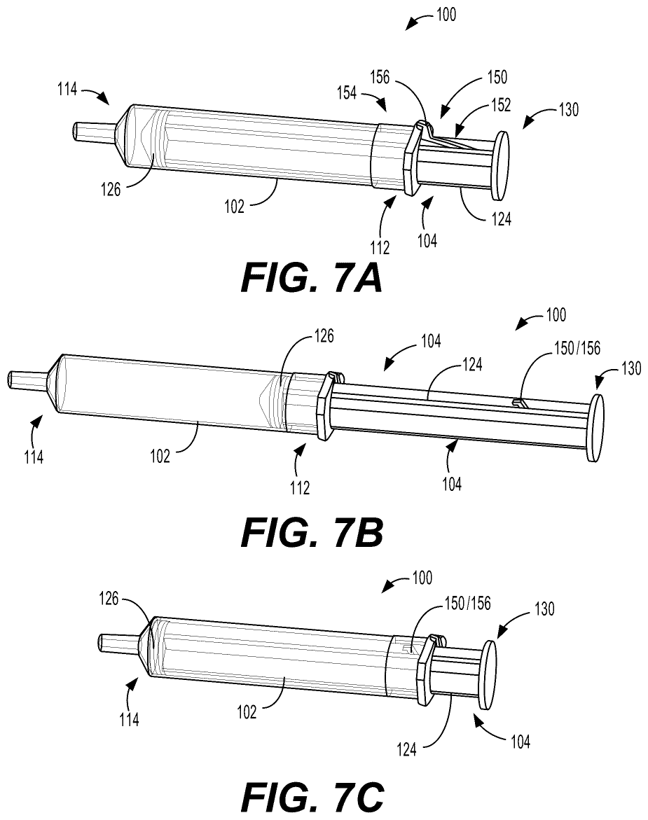

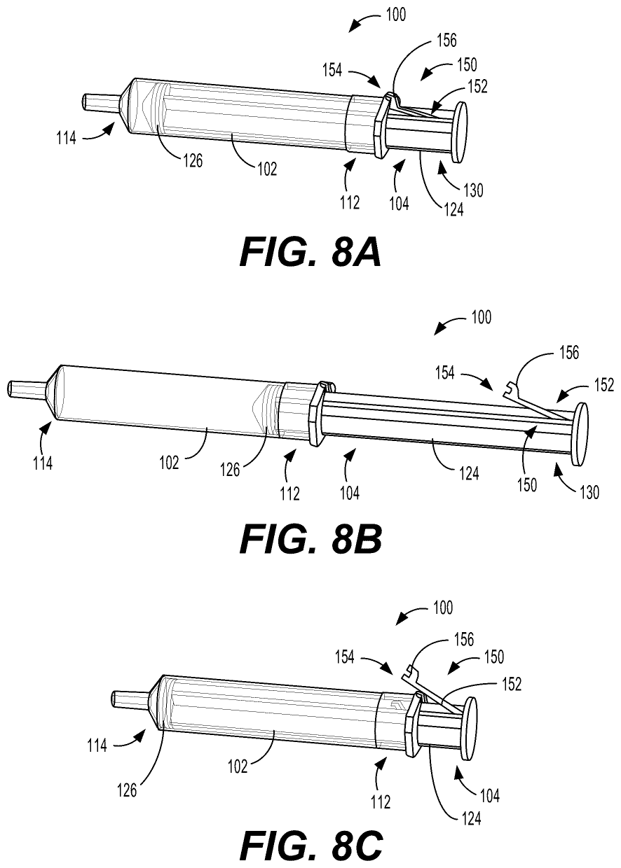

Referring now to FIGS. 7A-7C and FIGS. 8A-8C, and with continued reference to FIG. 2, a syringe 100 is shown according to additional embodiments of the disclosure. In order to provide for a pre-setting/pre-configuring of the syringe 100 to a desired collection volume and at a desired maximum vacuum pressure during blood draw, the plunger assembly 104 of syringe 100 includes a limiter 150 thereon that provides for setting of the plunger assembly 104 (and stopper 126 thereof) at a starting position that sets the initial volume at a desired amount. According to some embodiments, the limiter 150 is provided as a rotatable stop arm (hereafter “stop arm 150”) that is secured near the proximal end 130 of the plunger rod 124. The stop arm 150 includes a proximal end 152 and a distal end 154, with the proximal end 152 of the stop arm 150 affixed to the plunger rod 124 and the distal end 154 of the stop arm 150 including a hook or catch 156 configured to engage the proximal end 112 of the syringe barrel 102. In some embodiments, the proximal end 152 of stop arm 150 is secured to plunger rod 124 toward the proximal end 130 thereof and at a location between adjacent vanes/walls 134, so as to be seated therebetween.

The stop arm 150 may be formed integrally as part of the plunger rod 124 (i.e., as part of the same molded component) or may be secured thereto via an appropriate means of fastening. When formed integrally as part of the plunger rod 124, the stop arm 150 may be formed at an (initial) angle relative to the plunger rod 124, with the stop arm 150 being flexible to an angular position that is greater than or less than the initial angle. When provided as a separate component that is secured to the plunger rod 124, the stop arm 150 may be biased to a position where it is angled relative to the plunger rod 124, such as by a spring or other suitable biasing member.

In manufacturing and pre-setting/pre-configuring of the syringe 100, the stop arm 150 may initially be angled relative to plunger rod 124 so as to be aligned with the proximal end of the cylindrical outer wall 106 of syringe barrel 102, such that the catch 156 of stop arm 150 may engage the syringe barrel 102. The syringe 100 may thus be initially assembled by pushing the plunger assembly 104 distally into syringe barrel 102 until the catch 156 of stop arm 150 engages the proximal end 112 of the syringe barrel 102, with the stop arm 150 limiting the amount/length that plunger assembly 104 can be inserted into the syringe barrel 102 and defining the starting position of the plunger assembly 104 (and stopper 126). The stop arm 150 can be formed on plunger rod 124 and sized so that the plunger rod 124 is moved to a desired starting position, thereby setting the initial volume V1 at a desired amount.

During a subsequent use of the syringe barrel 102 in performing a blood draw, as the plunger assembly 104 is withdrawn proximally within syringe barrel 102, the catch 156 of stop arm 150 becomes disengaged from the proximal end 112 of the syringe barrel 102. Upon disengaging of the stop arm 150 from the syringe barrel 102, a biasing force of/on the stop arm 150 causes the stop arm 150 to rotate from its initial angle to a second angle that may be greater than or less than the initial angle.

In one embodiment, and as shown in the embodiment of FIGS. 7A-7C, the biasing of the stop arm 150 causes the stop arm 150 to rotate towards the plunger rod 124, so as to essentially lay flat against the plunger rod 124, such that the stop arm 150 is kept out of contact with the syringe barrel 102 when the plunger assembly 104 is pushed back distally into the syringe barrel 102 (during dispensing of the collected blood sample).

In another embodiment, and as shown in the embodiment of FIGS. 8A-8C, the biasing of the stop arm 150 causes the stop arm 150 to rotate outwards, so as to move away from the plunger rod 124, such that the stop arm 150 is kept out of contact with the syringe barrel 102 when the plunger assembly 104 is pushed back distally into the syringe barrel 102 (during dispensing of the collected blood sample).

For the syringe 100 as illustrated in FIGS. 7A-7C and FIGS. 8A-8C, performing of a blood draw may thus include steps of: (1) connecting the syringe 100 to the VAD 10, with the plunger assembly 104 pre-arranged at the first marking 142, and at the starting position (as limited by stop arm 150 and engaging thereof with proximal end 112 of syringe barrel 102), as shown in FIG. 7A and FIG. 8A; (2) pulling the plunger assembly 104 proximally to the second marking 144, and to the stop position, as shown in FIG. 7B and FIG. 8B, with the catch 156 of stop arm 150 disengaging the syringe barrel 102 and rotating inwardly/outwardly from its initial angle, to collect the blood sample; and (3) at the desired time, pushing the plunger assembly 104 distally to the distal end 114 of the syringe barrel 102 to dispense the blood sample to a specimen container, as shown in FIG. 7C and FIG. 8C, with the stop arm 150 positioned to enable the distal pushing of the plunger assembly 104.

According to additional embodiments of the disclosure, rather than the blood draw syringe itself being configured to provide for collection of a blood sample in a controlled manner to ensure that the maximum vacuum pressure within the syringe during a blood draw is controlled to a desired level (e.g., so that the maximum vacuum pressure is similar to a V acutainer pressure), a separate adapter may be provided for the syringe that provides similar control. That is, rather than the blood draw syringe 100 including features thereon that enable pre-positioning of the stopper within the syringe barrel 102 to provide a desired initial volume V1 in comparison to the final volume V2, as described in detail above, a separate adapter may be instead be employed that provides such an initial volume V1.

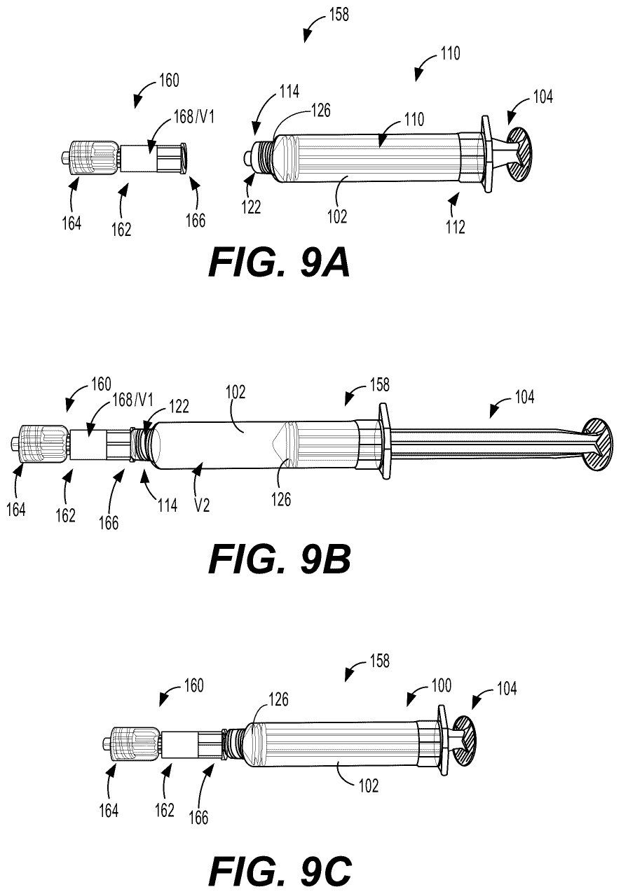

Referring to FIGS. 9A-9C, an adapter-syringe assembly 158 is illustrated that includes an adapter 160 connected to a blood draw syringe 100, in accordance with an aspect of the disclosure. The adapter 160 may be generally characterized as including an adapter body 162, a distal connector 164 at a distal end of the adapter body 162, and a proximal connector 166 at a proximal end of the adapter body 162. In some embodiments, the distal connector 164 of adapter 160 may comprise a male luer connector that may couple/mate with the (female) luer connection of the connector 42 of VAD 10, while the proximal connector 166 of adapter 160 may comprise a female luer connector that may couple/mate with the fluid connector component 122 (i.e., male luer connection) of the blood draw syringe 100.

The adapter body 162 encloses an internal volume 168 of the adapter 160. The adapter body 162 may be specifically constructed such that the internal volume 168 thereof is set at a (adapter) volume V1 that functions identical to the initial volume V1 described in the prior embodiments, with the adapter volume V1 chosen to match the volume of air within a Vacutainer for the targeted draw volume (i.e., a final volume V2). Thus, as non-limiting examples, the adapter volume V1 may be comprise 0.6 ml when used for a 7 mL draw and may comprise 3.6 ml when used for a 4 mL draw.

For the syringe 100 and adapter 160 as illustrated in FIGS. 9A-9C, performing of a blood draw may thus include steps of: (1) connecting the adapter 160 to the blood draw syringe 100, via mating of the proximal luer connector 166 of the adapter 160 with the luer connection 122 of the syringe, as shown in FIG. 9A; (2) connecting the adapter 160 and syringe 100 to the VAD (not shown), via mating of the distal luer connector 164 of the adapter 160 with the connector 42; (3) pulling the plunger assembly 104 proximally from a starting position (at the far distal end 114 of the syringe barrel 102) to a stop position, as shown in FIG. 9B, to collect the blood sample; and (4) at the desired time, pushing the plunger assembly 104 distally to the distal end 114 of the syringe barrel 102 to dispense the blood sample to a specimen container, as shown in FIG. 9C.

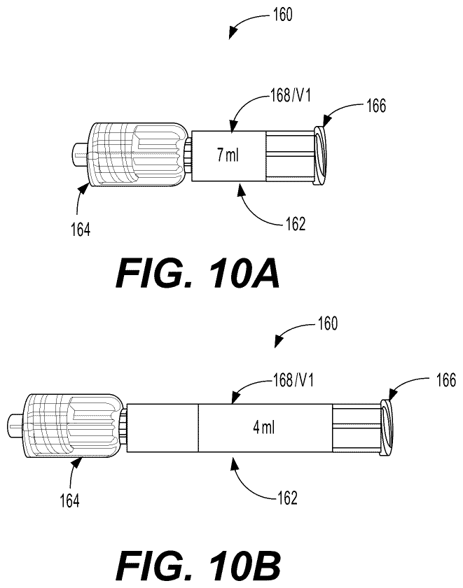

In some embodiments, and as shown in FIGS. 10A and 10B, the adapter 160 may be configured as an adjustable adapter 160, where the internal volume 168 within the adapter body 162 may be changed, such that the adapter 160 may enable various draw volumes by the syringe. That is, as described above, the adapter volume V1 may be chosen to match the volume of air within a Vacutainer for the targeted draw volume, such as the adapter volume V1 being set to 0.6 ml when used for a 7 mL draw and set to 3.6 ml when used for a 4 mL draw. To provide for such multiple adapter volumes V1, the adapter body 162 may comprise an extendable or expandable tube that may be shortened and/or lengthened to change the internal adapter volume V1. Thus, when the adapter 160 is to be used with a syringe to perform a 7 mL blood draw, the adapter body 162 may be used in a shortened configuration, such as shown in FIG. 10A, where the internal adapter volume V1 is 0.6 ml. When the adapter 160 is to be used with a syringe to perform a 4 mL blood draw, the adapter body 162 may be actuated to its lengthened configuration, such as shown in FIG. 10B, where the internal adapter volume V1 is increased to 3.6 ml.

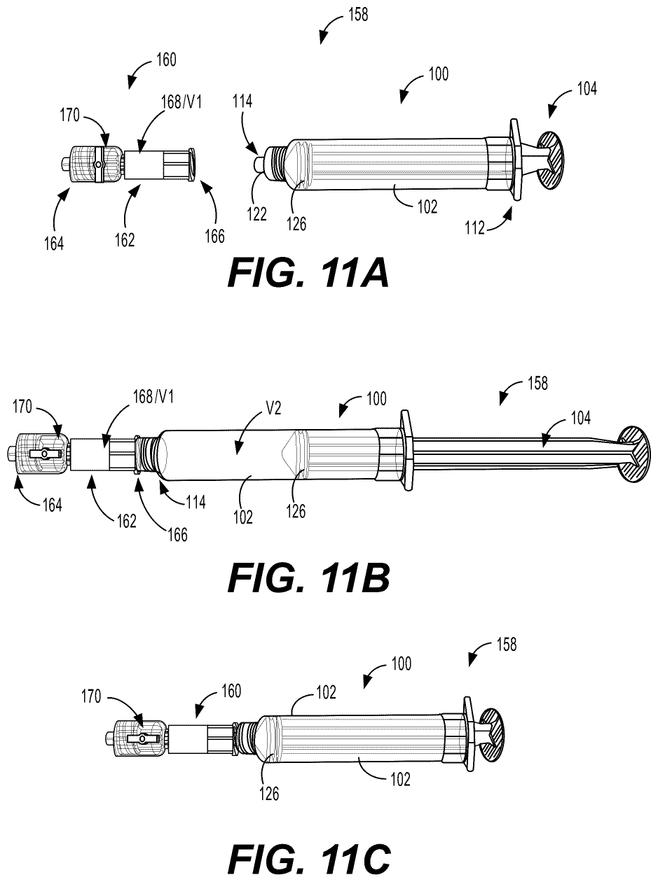

In some embodiments, and as shown in FIGS. 11A-11C, the adapter 160 may further include a stopcock 170 integrated therein that may be actuated between an open position and a closed position. The stopcock 170 may be in the closed position when the adapter 160 is initially connected to each of the syringe 100 and the VAD 10. With the stopcock 170 in the closed position, the syringe 100 the plunger assembly 104 may be drawn distally from a starting position (at the far distal end 114 of the syringe barrel 102) to a stop position, thereby creating/maintaining a maximum vacuum pressure possible within the syringe 100 before any blood flows into the syringe 100, which would reduce the maximum vacuum in the syringe 100 while blood is being drawn into the syringe barrel 102.

For the syringe 100 and adapter 160 as illustrated in FIGS. 11A-11C, performing of a blood draw may thus include steps of: (1) connecting the adapter 160 to the blood draw syringe 100 with the stopcock 170 in the closed position, as shown in FIG. 11A; (2) connecting the adapter 160 and syringe 100 to the VAD (not shown) with the stopcock 170 in the closed position; (3) pulling the plunger assembly 104 proximally from a starting position (at the far distal end 114 of the syringe barrel 102) to a stop position with the stopcock 170 in the closed position, and then actuating the stopcock 170 to the open position, to draw blood into the syringe 100, for collection of the blood sample, as shown in FIG. 11B; and (4) at the desired time, pushing the plunger assembly 104 distally to the distal end 114 of the syringe barrel 102 to dispense the blood sample to a specimen container, as shown in FIG. 11C.

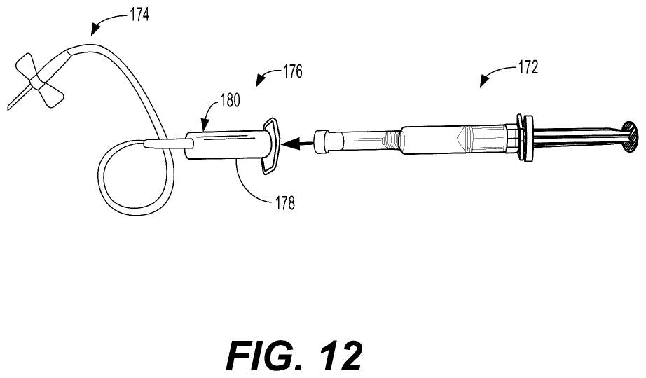

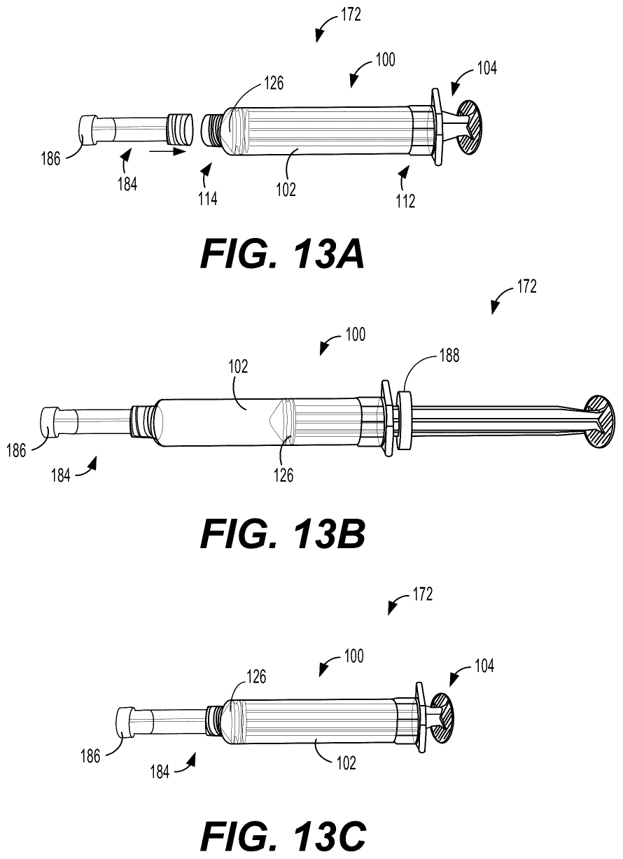

Referring now to FIG. 12 and FIGS. 13A-13C, an additional embodiment of the disclosure provides an adapter and blood draw syringe assembly 172 to be used with a blood collection set configured to accommodate a Vacutainer device. That is, as shown in FIG. 12, a VAD 174 may be provided that is generally similar in construction/arrangement to the VAD 10 of FIG. 1; however, rather than including a luer connector at a proximal end of an extension set as in the VAD 10, the VAD 174 of the embodiment of FIG. 12 may instead include a needle set 176 at the proximal end of an extension set 178. The needle set 176 may include a holder 178 having a hollow interior volume, along with a sharp-tipped needle 180 contained within the interior volume of the holder 178. The needle set 176 is configured to receive a standard vacuumed blood collection tube (i.e., Vacutainer) therein but, as described below, may also accommodate an adapter-syringe assembly 172 therein.

As shown in FIGS. 13A-13C, an adapter 182 may be provided that enables use of a blood draw syringe 100 with the VAD 174 and needle set 176 of FIG. 12. The adapter 182 may include an adapter body 184 that is constructed similar to the adapter body 162 illustrated in FIGS. 9A-9C but, rather than adapter 182 including a distal male luer connector thereon, the adapter 182 may instead include a rubber stopper or plug 186 that is operable with the needle set 176 of VAD. The plug 186 is seated at the distal end of the adapter body 184 to seal the distal end of the adapter body 184 and, with the adapter 182 connected to the blood draw syringe 100, the plug 186 enables the syringe 100 to function as a vacuumed blood collection tube (V acutainer). That is, with the adapter 182 connected to the blood draw syringe 100, the plunger assembly 104 may be retracted distally from a starting position (at the far distal end 114 of the syringe barrel 102) to a stop position corresponding to the desired draw volume, with retraction of the plunger assembly 104 creating a vacuum within the syringe barrel 102 that is maintained until a piercing of the plug 186 (by needle 180 of needle set 176) occurs. In some embodiments, a clip 188 (such as shown and described in detail in connection to FIGS. 5A-5C) may be provided on the syringe 100 to ensure that the plunger assembly 104 is retained at the stop position after retraction thereof.

For the syringe 100 as illustrated in FIGS. 13A-13C, performing of a blood draw may thus include steps of: (1) connecting the adapter 182 to the blood draw syringe 100, as shown in FIG. 13A; (2) pulling the plunger assembly 104 proximally from a starting position (at the far distal end 114 of the syringe barrel 102) to a stop position for the desired draw volume, as shown in FIG. 13B; (3) inserting the adapter 182 and a portion of the syringe 100 into the holder 178 of needle set 176 (not shown) and piercing the plug 186 with the needle 180, to draw blood into the syringe 100 via the vacuum within syringe barrel 102, for collection of the blood sample; and (4) at the desired time, pushing the plunger assembly 104 distally to the distal end 114 of the syringe barrel 102 to dispense the blood sample to a specimen container, as shown in FIG. 13C.

According to still additional embodiments of the disclosure, the structure of the plunger assembly 104 of blood draw syringe 100 may be modified to provide for collection of a blood sample in a controlled manner and ensure that the maximum vacuum pressure within the syringe during a blood draw is controlled to a desired level (e.g., so that the maximum vacuum pressure is similar to a Vacutainer pressure). That is, the structure of the plunger rod 124 and/or stopper 126 may be modified to help control/lower the maximum vacuum pressure within the syringe during a blood draw.

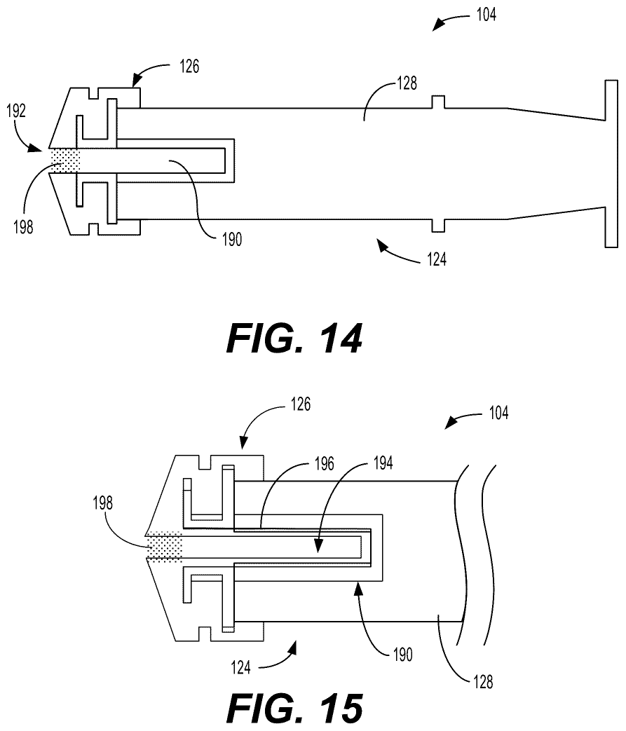

Referring to FIG. 14, a plunger assembly 104 is shown where each of the plunger rod 124 and stopper 126 has a construction that helps lower a maximum vacuum pressure within the syringe 100 during a blood draw. Specifically, the plunger rod 124 includes an elongate (air) chamber 190 formed in the main body 128 thereof, at the distal end 132 of the main body 128, while the stopper 126 includes a channel 192 formed therein that extends lengthwise through the stopper 126-with the channel 192 centered on the stopper 126 so as to be aligned with the elongate chamber 190 in plunger rod 124.

Referring to FIG. 15, in another embodiment similar to the plunger assembly 104 of FIG. 14, rather than the plunger rod 124 defining an air chamber, the stopper 126 may define an (air) chamber 194. That is, while plunger rod 124 (i.e., main body 128 thereof) still defines an elongate chamber 190 therein, the stopper 126 is configured to include a protrusion 196 that extends proximally out therefrom and into the chamber 190 of the plunger rod 124. The protrusion 196 comprises a hollow protrusion with a closed proximal end that defines the chamber 194 for the plunger assembly 104.

In some embodiments, and for either of the embodiments of FIGS. 14 and 15, a vent plug 198 may be secured within the channel (192 or chamber 194) formed in stopper 126. The vent plug 198 may be secured at a distal end of the channel/chamber, so as to form part of a distal face of the stopper 126. The vent plug 198 may be formed of a material that is air permeable, but that is impervious to fluids (e.g., blood) passing therethrough. The vent plug 198 thus allows for air movement within the syringe barrel 102, in order to reduce the maximum vacuum pressure in syringe 100 during a blood draw, while not allowing blood to enter into and become trapped in the chamber 190 of plunger rod 124 (or chamber 194 of stopper 126).

As previously described above for various embodiments, including the embodiment of FIGS. 3A-3C, the maximum vacuum pressure provided in syringe 100 during a blood draw may be set/controlled via positioning of the stopper 126 at a desired initial location, i.e., setting the initial volume V1, according to: P2=P1*V1/V2. Knowing a desired P2 value, an initial volume V1 may be selected that ensures that P2 is set at a desired value, with the air chamber 190 in the plunger rod 124 (or the air chamber 194 in stopper 126) contributing to the overall initial volume V1. Thus, knowing the relationship between pressure and volume, a pre-setting of the initial volume V1 in syringe 100 can ensure that the maximum vacuum pressure within the syringe 100 during a blood draw is controlled to a desired level. In some embodiments, the maximum vacuum pressure P2 may be controlled so that it matches the maximum vacuum pressure that is set/provided within a vacuumed blood collection tube (i.e., a Vacutainer) of similar draw volume, as described in detail previously.

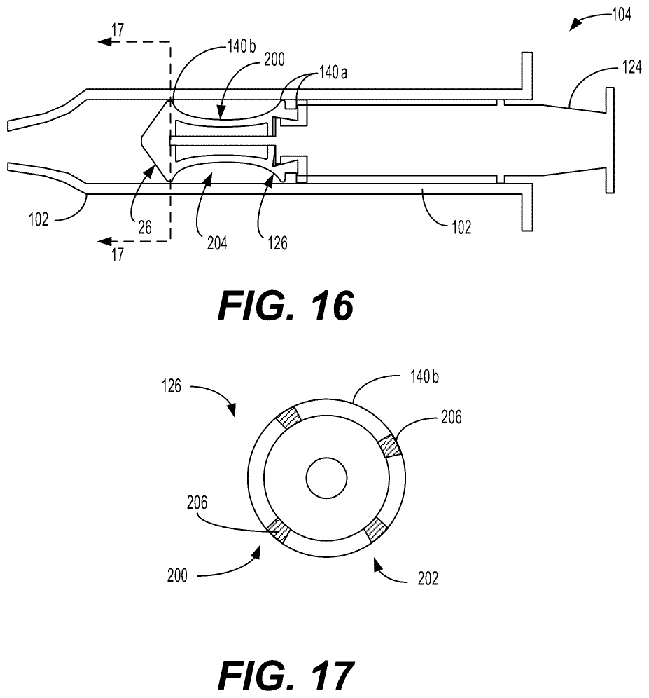

Referring now to FIGS. 16 and 17, a plunger assembly 104 is shown where stopper 126 has a construction that helps lower a maximum vacuum pressure within the syringe 100 during a blood draw. Specifically, the stopper 126 has an elongate construction where a proximal group of annular flanges or ribs 140a is separated longitudinally from a distal annular flange or rib 140b by an inter-rib gap 200. The proximal ribs 140a are configured as annular flanges configured to form a tight seal with the cylindrical outer wall 106 of syringe barrel 102. The distal rib 140b is configured as an annular flange that includes a plurality of channels 202 formed therein, arranged circumferentially about the rib 140b.

According to embodiments of the disclosure, with the stopper 126 having an elongate construction where the proximal ribs 140a are separated from the distal rib 140b by the inter-rib gap 200, an air pocket 204 is formed in an area/volume defined between the syringe barrel 102 and the inter-rib gap region of stopper 126. This air pocket 204 is fluidly connected with the proximal portion of the chamber 110 in syringe barrel 102 via the channels 202 formed in the distal rib 140b.

In some embodiments, a venting material 206 may be provided in each of the channels 202 formed in distal rib 140b. The venting material 206 may be formed of a material that is air permeable, but that is impervious to fluids (e.g., blood) passing therethrough, so as to allow for air movement within the syringe barrel 102, in order to reduce the maximum vacuum pressure in syringe 100 during a blood draw, while not allowing blood to enter into and become trapped in the air pocket 204.

As previously described above the maximum vacuum pressure provided in syringe 100 during a blood draw may be set/controlled via positioning of the stopper 126 at a desired initial location, i.e., setting the initial volume V1, according to: P2=P1*V1/V2. Knowing a desired P2 value, an initial volume V1 may be selected that ensures that P2 is set at a desired value, with the air pocket 204 provided by the elongate stopper 126 contributing to the overall initial volume V1. Thus, knowing the relationship between pressure and volume, a pre-setting of the initial volume V1 in syringe 100 can ensure that the maximum vacuum pressure within the syringe 100 during a blood draw is controlled to a desired level. In some embodiments, the maximum vacuum pressure P2 may be controlled so that it matches the maximum vacuum pressure that is set/provided within a vacuumed blood collection tube (i.e., a V acutainer) of similar draw volume, as described in detail previously.

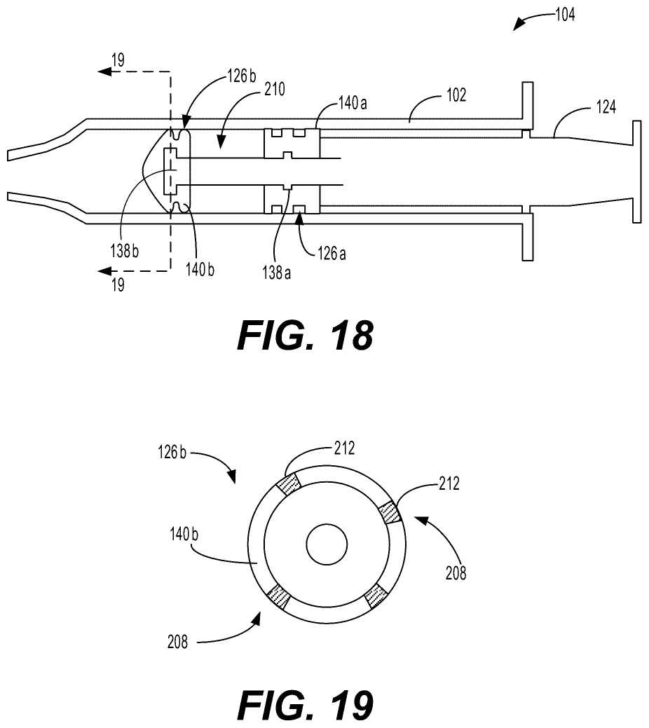

Referring now to FIGS. 18 and 19, a plunger assembly 104 is shown having a construction that helps lower a maximum vacuum pressure within the syringe 100 during a blood draw. Specifically, the plunger assembly 104 is configured to include a proximal stopper 126a and a distal stopper 126b, with each of the stoppers 126a, 126b secured to a plunger rod 124 via a first flanged member 138a and a second flanged member 138b, respectively. As shown in FIG. 18, the flanged members 138a, 138b are longitudinally spaced apart on the main body 128 of plunger rod 124, such that the proximal stopper 126a and distal stopper 126b will also be longitudinally spaced apart when secured to the plunger rod 124. The proximal stopper 126a includes annular flanges or ribs 140a thereon that are configured to form a tight seal with the cylindrical outer wall 106 of syringe barrel 102. The distal stopper 126b includes one or more annular flanges or ribs 140b thereon that are configured to include a plurality of channels 208 formed therein, arranged circumferentially about the rib(s) 140b, as previously described for the distal rib 140 in the embodiment of FIGS. 16 and 17.

According to embodiments of the disclosure, with the proximal stopper 126a and distal stopper 126b being longitudinally spaced apart on plunger rod 124, an air pocket 210 is formed in an area/volume defined between the syringe barrel 102 and stoppers 126a, 126b. This air pocket 210 is fluidly connected with the proximal portion of the chamber 100 in syringe barrel 102 via the channels 208 formed in the distal rib 140b.

In some embodiments, a venting material 212 may be provided in each of the channels 208 formed in the rib(s) 140b of distal stopper 126b. The venting material 212 may be formed of a material that is air permeable, but that is impervious to fluids (e.g., blood) passing therethrough, so as to allow for air movement within the syringe barrel 102, in order to reduce the maximum vacuum pressure in syringe 100 during a blood draw, while not allowing blood to enter into and become trapped in the air pocket 210.

As previously described above the maximum vacuum pressure provided in syringe 100 during a blood draw may be set/controlled via positioning of the stopper 126 at a desired initial location, i.e., setting the initial volume V1, according to: P2=P1*V1/V2. Knowing a desired P2 value, an initial volume V1 may be selected that ensures that P2 is set at a desired value, with the air pocket 210 provided by the spaced apart stoppers 126a, 126b contributing to the overall initial volume V1. Thus, knowing the relationship between pressure and volume, a pre-setting of the initial volume V1 in syringe 100 can ensure that the maximum vacuum pressure within the syringe 100 during a blood draw is controlled to a desired level. In some embodiments, the maximum vacuum pressure P2 may be controlled so that it matches the maximum vacuum pressure that is set/provided within a vacuumed blood collection tube (i.e., a Vacutainer) of similar draw volume, as described in detail previously.

Beneficially, embodiments of the disclosure thus provide a blood draw syringe and optional adapter useable with a VAD for collection of a blood sample. In some embodiments, the blood draw syringe is configured to be pre-set/pre-configured with the stopper thereof at a desired location prior to initiation of a blood draw sequence, with the positioning of the stopper providing a pre-set volume that functions to control a vacuum pressure within the syringe during the blood draw, such as by limiting the vacuum pressure to the level of a Vacutainer draw. In other embodiments, a plunger assembly of the blood draw syringe may be configured to provide an additional air pocket that functions to control a vacuum pressure within the syringe during the blood draw, such as by limiting the vacuum pressure to the level of a Vacutainer draw. In still other embodiments, an adapter is connected to the blood draw syringe to provide a pre-set volume that functions to control a vacuum pressure within the syringe during the blood draw. In still other embodiments, the adapter may operate to configure the blood draw syringe as a vacuumed blood collection tube that is useable with standard blood collection needle sets typically used with vacuumed blood collection tubes. The blood draw syringe and/or associated adapter eliminates the impact of syringe draw rate on hemolysis, thus making the blood draw syringe more user friendly and standardizing the blood draw procedure.

While several embodiments of a system configured for blood collection were described in the foregoing detailed description, those skilled in the art may make modifications and alterations to these embodiments without departing from the scope and spirit of the invention. That is, it is recognized that features of each system of each embodiment may be incorporated into the system of other embodiments. Accordingly, the foregoing description is intended to be illustrative rather than restrictive. The invention described hereinabove is defined by the appended claims and all changes to the invention that fall within the meaning and the range of equivalency of the claims are embraced within their scope.

The invention claimed is

1. A blood draw syringe connectable to a vascular access device for performing a blood draw, the blood draw syringe comprising:
a syringe barrel having a distal end and a proximal end and that defines a chamber therein, and a fluid connector component at the distal end of the syringe barrel; and
a plunger assembly including a plunger rod having a proximal end and a distal end, and a stopper positioned at the distal end of the plunger rod that is insertable into the proximal end of the syringe barrel, wherein the plunger assembly is moveable within the syringe barrel to move the stopper between a starting position and a stop position, with a first volume provided in the chamber between the stopper and the distal end of the syringe barrel with the stopper at the starting position and a second volume provided in the chamber between the stopper and the distal end of the syringe barrel with the stopper at the stop position, the second volume being greater than the first volume;
wherein the syringe barrel includes:
a first marking provided on the syringe barrel indicating the starting position of the stopper; and
a second marking provided on the syringe barrel indicating the stop position of the stopper; and
wherein, with the plunger assembly moving proximally within the syringe barrel during a blood draw to move the stopper from the starting position to the stop position, a size of the first volume limits a vacuum pressure in the syringe barrel during the blood draw.

2. The blood draw syringe of claim 1, wherein the first marking comprises:
a first sub-marking provided on the syringe barrel indicating a first starting position of the stopper; and
a second sub-marking provided on the syringe barrel indicating a second starting position of the stopper.

3. The blood draw syringe of claim 1, further comprising a clip attached to the plunger rod at a location that, when the clip abuts the proximal end of the syringe barrel, the stopper is at the starting position and aligned with the first marking.

4. The blood draw syringe of claim 3, wherein the clip is configured to be removable from the plunger rod, to enable a distal advancement of the plunger assembly fully into the syringe barrel.

5. The blood draw syringe of claim 1, further comprising a stopcock provided in the syringe barrel and positioned at or adjacent the fluid connector component at the distal end of the syringe barrel, the stopcock actuatable between an open position and a closed position, to control a flow of fluid into the chamber of the syringe barrel.

6. The blood draw syringe of claim 1, wherein the first marking and the second marking comprise one or more of a line, a symbol, a picture, an icon, an alphabetical descriptor, or a numeric descriptor.

7. The blood draw syringe of claim 1, wherein a longitudinal positioning of the first marking and the second marking on the syringe barrel that indicate the starting position and the stop position of the stopper, respectively, are selected to set the first volume and the second volume and, in turn, set a maximum desired vacuum pressure in the syringe barrel during the blood draw.

8. The blood draw syringe of claim 7, wherein the maximum desired vacuum pressure is a vacuum pressure equal to or less than a vacuum pressure in a vacuumed blood collection tube having a same draw volume as a draw volume of the blood draw syringe.

9. A blood draw syringe connectable to a vascular access device for performing a blood draw, the blood draw syringe comprising:
a syringe barrel having a distal end and a proximal end and that defines a chamber therein, and a fluid connector component at the distal end of the syringe barrel; and
a plunger assembly including a plunger rod having a proximal end and a distal end, and a stopper positioned at the distal end of the plunger rod that is insertable into the proximal end of the barrel, wherein the plunger assembly is moveable within the syringe barrel to move the stopper between a starting position and a stop position, with a first volume provided in the chamber between the stopper and the distal end of the syringe barrel with the stopper at the starting position and a second volume provided in the chamber between the stopper and the distal end of the syringe barrel with the stopper at the stop position, the second volume being greater than the first volume;
wherein the plunger rod includes a stop arm affixed thereto at or adjacent the distal end of the plunger rod, the stop arm including:
a proximal end affixed to the plunger rod; and
a free distal end having a catch formed thereon configured to engage the proximal end of the syringe barrel when the plunger assembly is in the starting position;
wherein the stop arm is at an initial angle when engaged with the proximal end of the syringe barrel, and wherein the stop arm is configured to rotate to a second angle when disengaged from the proximal end of the syringe barrel.

10. The blood draw syringe of claim 9, wherein with the plunger assembly in the starting position, the stop arm prevents further distal advancement of the plunger assembly into the syringe barrel.

11. The blood draw syringe of claim 9, wherein the catch is disengaged from the proximal end of the syringe barrel upon a proximal movement of the plunger assembly.

12. The blood draw syringe of claim 9, wherein with the stop arm rotated to the second angle, the plunger assembly may be distally advanced into the syringe barrel, until the stopper contacts the distal end of the syringe barrel.

13. The blood draw syringe of claim 9, wherein when rotated to the second angle, the stop arm is rotated toward the plunger rod or rotated away from the plunger rod.

14. The blood draw syringe of claim 9, wherein with the plunger assembly moving proximally within the syringe barrel during a blood draw to move the stopper from the starting position to the stop position, a size of the first volume limits a vacuum pressure in the syringe barrel during the blood draw.

15. A system for performing a blood draw, the system comprising:
a vascular access device including:
a catheter comprising a catheter distal end and a catheter proximal end;
a catheter adapter including a distal adapter end and a proximal adapter port and defining a lumen therein, wherein the catheter proximal end is secured to the distal adapter end; and
an extension set extending from the catheter adapter and in fluid communication with the catheter via the lumen of the catheter adapter, the extension set comprising an extension tube having a distal end coupled to the catheter adapter and a proximal end having a connector thereon; and
a syringe connected to the vascular access device, the syringe comprising:
a syringe barrel having a distal end and a proximal end and that defines a chamber therein, and a fluid connector component at the distal end of the barrel that is configured to engage the connector, to fluidly connect the syringe to the vascular access device; and
a plunger assembly including a plunger rod having a proximal end and a distal end, and a stopper positioned at the distal end of the plunger rod that is insertable into the proximal end of the barrel, wherein the plunger assembly is moveable within the syringe barrel between a starting position and a stop position, with a first volume provided in the chamber between the stopper and the distal end of the syringe barrel with the stopper at the starting position and a second volume provided in the chamber between the stopper and the distal end of the syringe barrel with the stopper at the stop position, the second volume being greater than the first volume;
wherein the syringe is configured to enable pre-setting of the plunger assembly within the syringe barrel prior to a blood draw, with the stopper positioned at the starting position, thereby limiting a vacuum pressure in the syringe barrel during the blood draw.

16. The system of claim 15, wherein the syringe barrel includes:
a first marking provided on the syringe barrel indicating the starting position of the stopper; and
a second marking provided on the syringe barrel indicating the stop position of the stopper;
wherein, with the plunger assembly moving proximally within the syringe barrel during a blood draw to move the stopper from the starting position to the stop position, a size of the first volume limits a vacuum pressure in the syringe barrel during the blood draw.

17. The system of claim 16, wherein the syringe further comprises a clip attached to the plunger rod at a location that, when the clip abuts the proximal end of the syringe barrel, the stopper is at the starting position and aligned with the first marking, and wherein the clip is removable from the plunger rod, to enable a distal advancement of the plunger assembly fully into the syringe barrel.

18. The system of claim 15, wherein the syringe further comprises a stopcock provided in the syringe barrel and positioned at or adjacent the fluid connector component at the distal end of the syringe barrel, the stopcock actuatable between an open position and a closed position, to control a flow of fluid into the chamber of the syringe barrel.

19. The system of claim 15, wherein the plunger rod includes a stop arm affixed thereto at or adjacent the distal end of the plunger rod, the stop arm including:
a proximal end affixed to the plunger rod; and
a free distal end having a catch formed thereon configured to engage the proximal end of the syringe barrel when the plunger assembly is in the starting position;
wherein the stop arm is at an initial angle when engaged with the proximal end of the syringe barrel, and wherein the stop arm is configured to rotate to a second angle when disengaged from the proximal end of the syringe barrel.

20. The system of claim 19, wherein with the plunger assembly in the starting position, the stop arm prevents further distal advancement of the plunger assembly into the syringe barrel, and with the stop arm rotated to the second angle, the plunger assembly may be distally advanced into the syringe barrel until the stopper contacts the distal end of the syringe barrel, with the stop arm rotating to the second angle when the plunger assembly is moved proximally from the starting position toward the stop position.
